(19) United States
(12) Patent Application Publication
(10) Pub. No.: US 2015/0184259 A1
(43) Pub. Date: Jul. 2, 2015

(54) PROCESSES AND APPARATUS FOR PRODUCING FERMENTABLE SUGARS FROM BIOMASS BY HOT-WATER EXTRACTION AND ENZYMATIC HYDROLYSIS

(71) Applicant: API Intellectual Property Holdings, LLC, Atlanta, GA (US)

(72) Inventors: Ryan ZEBROSKI, Fayetteville, GA (US); James SCHULTZE, Alpena, MI (US)

(21) Appl. No.: 14/583,566

(22) Filed: Dec. 26, 2014

Related U.S. Application Data
(60) Provisional application No. 61/921,087, filed on Dec. 27, 2013.

(51) Int. Cl. C13K 1/02; C12P 19/02; C13K 1/04; C12P 7/10; C12P 19/14

(52) U.S. Cl. CPC C13K 1/02; C12P 7/10; C12P 19/14; C13K 1/04; C12P 19/02; C12P 2201/00

(57) ABSTRACT

The present invention is capable of producing glucose and hemicellulose sugars from lignocellulosic biomass. In some variations, a process includes optionally pre-steaming a biomass feedstock; extracting the feedstock with liquid hot water to produce glucan-rich solids and an extract liquor containing dissolved solids, which include hemicellulosic oligomers and lignin; washing the glucan-rich solids; hydrolyzing the hemicellulosic oligomers by contacting the extract liquor with an acid catalyst or enzymes possessing hemicellulase activity; separately hydrolyzing the glucan by contacting the glucan-rich solids stream with an acid catalyst or enzymes possessing glucanase activity, optionally with removing the glucose in situ by microfiltration and/or ultrafiltration; and recovering or fermenting each of the hemicellulosic monomers and the glucose. Preferred configurations and conditions are disclosed.

### PRIORITY DATA

This patent application is a non-provisional application claiming priority to U.S. Provisional Patent App. No. 61/921,087, filed Dec. 27, 2013, which is hereby incorporated by reference herein.

### FIELD OF THE INVENTION

The present invention generally relates to processes for recovering fermentable sugars from lignocellulosic biomass.

### BACKGROUND OF THE INVENTION

There is a worldwide interest in finding replacements or substitutes for crude oil and other fossil fuels. For a variety of reasons, ethanol is currently being produced commercially largely from food feedstocks such as corn, wheat, and sugarcane. Ethanol may also be produced from biomass, which is considered to be any naturally occurring organic material containing cellulose.

Biomass contains cellulose and hemicellulose which may be converted into C₆ sugars such as glucose and C₅ sugars such as xylose. The structure of these materials in biomass may be considered as a long strand of crystalline cellulose surrounded by a layer of hemicellulose with both the cellulose and hemicellulose surrounded by lignin. Hemicelluloses are generally linear or branched polymers of C₅ sugars, but may include other compounds such as acetyl groups that form acetic acid in solution.

For biomass processing to fermentable sugars, the effective pretreatment of biomass is critical to exposing the cellulose to enzymatic hydrolysis. Many biomass pretreatment technologies exist. It is typical for pretreatment processes to involve an initial mechanical step in which biomass is comminuted by a combination of chipping, grinding, and/or milling. For instance, steam explosion processes use explosive decompression to significantly reduce the particle size of coarse biomass, whereas other pretreatment processes commonly employ a secondary grinding or milling step to further reduce the particle size of the chipped biomass. Dilute-acid hydrolysis is another common pretreatment process.

Improvements are still needed in lignocellulosic biomass processes, in order to economically produce fermentable sugars from both the cellulose and hemicellulose fractions.

### SUMMARY OF THE INVENTION

The present invention addresses the aforementioned needs in the art.

In some variations, the present invention provides a process for producing fermentable sugars from cellulosic biomass, the process comprising:

(a) providing a feedstock comprising cellulosic biomass;

(b) optionally pre-steaming the feedstock;

(c) extracting the feedstock with liquid hot water under effective extraction conditions to produce cellulose-rich solids and an extract liquor containing dissolved solids, wherein the dissolved solids include hemicellulosic oligomers and lignin;

(d) washing the cellulose-rich solids to produce a washed cellulose-rich solids stream comprising glucan, wherein the washing is carried out at a washing pH selected from about 3 to about 7, and wherein the washing removes hydrolysis inhibitors from the cellulose-rich solids;

(e) hydrolyzing the hemicellulosic oligomers to hemicellulosic monomers by contacting the extract liquor with an acid catalyst or enzymes possessing hemicellulase activity;

(f) hydrolyzing the glucan to glucose, separately from step (e), by contacting the cellulose-rich solids stream with an acid catalyst or enzymes possessing glucanase activity; and

(g) recovering, fermenting, and/or further processing each of the hemicellulosic monomers and the glucose, separately or in combination.

In some embodiments, effective extraction conditions include an extraction temperature selected from about 160° C. to about 220° C., and an extraction time selected from about 3 minutes to about 4 hours. In certain embodiments, effective extraction conditions include an extraction temperature selected from about 160° C. to about 200° C. or from about 170° C. to about 185° C., and an extraction time selected from about 3 minutes to about 30 minutes or from about 10 minutes to about 20 minutes.

In some embodiments, extracting in step (c) employs an acid catalyst, which may be an organic acid or an inorganic acid. For example, the acid catalyst may be acetic acid, formic acid, lactic acid, or another organic acid. In some embodiments, the acid catalyst is sulfuric acid, sulfurous acid, sulfur dioxide, or a combination thereof.

The hydrolysis inhibitors removed during step (d) may include, but are not limited to, one or more compounds selected from the group consisting of acetic acid, formic acid, lactic acid, furfural, hydroxymethylfurfural, hemicellulose oligomers, and combinations, derivatives, or degradation products thereof. The washing in step (d) generates a wash liquid that may be combined with the extract liquor, or separately processed or purged.

In some embodiments, the washing pH is selected from about 5 to about 6. In certain embodiments, the washing pH is selected from about 5.3 to about 5.6. In certain preferred embodiments, step (d) employs countercurrent washing.

Step (g) may include fermentation of the glucose to any fermentation product. In some embodiments, step (g) comprises fermentation of the hemicellulosic monomers to any fermentation product which may be the same or different than the glucose-fermentation product. In some embodiments, step (g) comprises concentration of the glucose and/or the hemicellulosic monomers by evaporation, membrane filtration, or another suitable operation.

Optionally, the process further comprises removing at least a portion of the lignin, in dissolved form, from the extract liquor. Optionally, the process further comprises removing lignin that is present in suspended form from the extract liquor.

Other variations of the invention provide a process for producing fermentable sugars from cellulosic biomass, the process comprising:

(a) providing a feedstock comprising cellulosic biomass;

(b) optionally pre-steaming the feedstock;

(c) extracting the feedstock with liquid hot water under effective extraction conditions to produce cellulose-rich solids and an extract liquor containing dissolved solids, wherein the dissolved solids include hemicellulosic oligomers and lignin;

(d) washing the cellulose-rich solids to produce a washed cellulose-rich solids stream comprising glucan;

(e) hydrolyzing the hemicellulosic oligomers to hemicellulosic monomers by contacting the extract liquor with an acid catalyst or enzymes possessing hemicellulase activity;

(f) hydrolyzing the glucan to glucose by contacting the cellulose-rich solids stream with an acid catalyst or enzymes possessing glucanase activity, wherein step (f) includes removing the glucose in situ by microfiltration and/or ultrafiltration; and

(g) recovering, fermenting, and/or further processing each of the hemicellulosic monomers and the glucose, separately or in combination.

In some embodiments, effective extraction conditions include an extraction temperature selected from about 160° C.-220° C., about 160-200l° C., or about 170-185° C. and an extraction time selected from about 3 minutes to about 4 hours, about 3-30 minutes, or about 10-20 minutes. An organic or inorganic acid catalyst may be utilized to aid extraction.

In some embodiments, washing in step (d) removes hydrolysis inhibitors from the cellulose-rich solids and generates a wash liquid that is combined with the extract liquor. The hydrolysis inhibitors may include one or more compounds selected from the group consisting of acetic acid, formic acid, lactic acid, furfural, hydroxymethylfurfural, hemicellulose oligomers, and combinations, derivatives, or degradation products thereof. Washing may be performed at a washing pH is selected from about 3 to about 7, or about 5 to about 6 in certain embodiments. Washing may be performed countercurrently.

Steps (e) and (f) may be performed separately or in a combined process step or unit. In some embodiments, the microfiltration and/or ultrafiltration generates a retentate that is returned to step (f) to recycle the acid catalyst or the enzymes possessing glucanase activity.

In some embodiments, step (g) comprises fermentation of the glucose and/or fermentation of the hemicellulosic monomers. Step (g) may include concentration of the glucose and/or the hemicellulosic monomers by evaporation, membrane filtration, and/or another unit operation. The process may further include lignin removal and recovery.

### BRIEF DESCRIPTION OF THE FIGURE

FIG. 1 is an exemplary block-flow diagram of the process of the invention, in some embodiments.

### DETAILED DESCRIPTION OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

The biomass feedstock may be selected from hardwoods, softwoods, forest residues, agricultural residues (such as sugarcane bagasse or straw), industrial wastes, consumer wastes, or combinations thereof.

In some variations, the present invention provides a process for producing fermentable sugars from cellulosic biomass, the process comprising:

(a) providing a feedstock comprising cellulosic biomass;

(b) optionally pre-steaming the feedstock;

(c) extracting the feedstock with liquid hot water under effective extraction conditions to produce cellulose-rich solids and an extract liquor containing dissolved solids, wherein the dissolved solids include hemicellulosic oligomers and lignin;

(d) washing the cellulose-rich solids to produce a washed cellulose-rich solids stream comprising glucan, wherein the washing is carried out at a washing pH selected from about 3 to about 7, and wherein the washing removes hydrolysis inhibitors from the cellulose-rich solids;

(e) hydrolyzing the hemicellulosic oligomers to hemicellulosic monomers by contacting the extract liquor with an acid catalyst or enzymes possessing hemicellulase activity;

(f) hydrolyzing the glucan to glucose, separately from step (e), by contacting the cellulose-rich solids stream with an acid catalyst or enzymes possessing glucanase activity; and

(g) recovering, fermenting, and/or further processing each of the hemicellulosic monomers and the glucose, separately or in combination.

In some embodiments, effective extraction conditions include an extraction temperature selected from about 160° C. to about 220° C., and an extraction time selected from about 3 minutes to about 4 hours. In certain embodiments, effective extraction conditions include an extraction temperature selected from about 160° C. to about 200° C. or from about 170° C. to about 185° C., and an extraction time selected from about 3 minutes to about 30 minutes or from about 10 minutes to about 20 minutes.

In some embodiments, extracting in step (c) employs an acid catalyst, which may be an organic acid or an inorganic acid. For example, the acid catalyst may be acetic acid, formic acid, lactic acid, or another organic acid. In some embodiments, the acid catalyst is sulfuric acid, sulfurous acid, sulfur dioxide, or a combination thereof.

Known reactors or related equipment may be employed for the extraction step. The reactor (or extraction vessel) may be batch or continuous, and it may be a stirred reactor, a plug-flow reactor, or a different flow pattern. A plurality of reactors may be employed.

The hydrolysis inhibitors removed during step (d) may include, but are not limited to, one or more compounds selected from the group consisting of acetic acid, formic acid, lactic acid, furfural, hydroxymethylfurfural, hemicellulose oligomers, and combinations, derivatives, or degradation products thereof. The washing in step (d) generates a wash liquid that may be combined with the extract liquor, or separately processed or purged.

In some embodiments, removing at least a portion of the hemicellulose oligomers (e.g., xylose oligomers) allows for a more effective and efficient action of glucanase enzymes on the remaining glucan. It may be possible to reduce the thermal pretreatment severity (of the extraction conditions), resulting in a higher xylose yield, and less loss to furfural, when washing is performed to remove hemicellulose oligomers.

In some embodiments, the washing pH is selected from about 5 to about 6, such as about 5.0, 5.1, 5.2, 5.3, 5.4, 5.5, 5.6, 5.7, 5.8, 5.9, or 6.0. In some embodiments, the washing pH is selected from about 6 to about 7, such as about 6.0, 6.1, 6.2, 6.3, 6.4, 6.5, 6.6, 6.7, 6.8, 6.9, or 7.0. In certain embodiments, the washing pH is selected from about 5.3 to about 5.6, which is a range of pH that may be readily achieved with process control. Preferably, the washing pH is selected so that neutralization of the biomass takes place during washing. The purpose of the neutralization is to bring the entire biomass particle, including the internal pore structure, to a pH which will not adversely affect the enzyme, which it will encounter in the subsequent hydrolysis step.

Washing pH may vary during washing, as components enter solution. Optionally, one or more additives may be introduced to enhance washing efficiency. The additives may be components recycled from downstream operations.

In certain preferred embodiments, step (d) employs countercurrent washing. A countercurrent wash may be performed to remove inhibitors found in the dissolved solids fraction of the pretreated material, such as xylose oligomers. A countercurrent scheme may create a hemicellulose-rich stream and a glucan-rich stream, each of which may be hydrolyzed separately with enzymes formulated for the specific composition of the stream. The efficiency of the wash may be rated by measuring the amount of dissolved solids remaining in the material. The wash liquid will typically contain hemicellulose oligomers and monomers, glucose and oligomers of glucose (e.g., cellobiose), acetic acid, and various minor species.

Any known equipment may be employed for washing. In some embodiments, the washing apparatus includes a slurry mix tank, followed by a screw press with wash capability, followed by a second slurry mix tank, followed by a second screw press with wash capability. Washing may be batch, continuous, or semi-continuous.

Step (f) to hydrolyze the glucan to glucose is preferably performed (in some embodiments) separately from step (e). Typically, the hemicellulose-rich stream produced by washing is largely dissolved solids, with a small amount of residual suspended solids. The hydrolysis of this hemicellulose-rich material may be accomplished, for example, in an agitated, pH-controlled and temperature-controlled hydrolyzer, separate from the hydrolysis of the suspended solids glucan-rich fraction of the wash. The isolation and independent hydrolysis of the hemicellulosic oligomers helps in the conversion of both the hemicellulosic oligomers, as well as the cellulose-rich solids stream. The hemicellulosic oligomers can be more efficiently converted by the application of an enzyme blend formulated for the conversion of xylose oligomers (or mannose oligomers, for example, in the case of softwoods). The glucanase applied to the cellulose-rich solids stream is not inhibited by the presence of hemicellulosic oligomers.

The glucan-rich stream produced by the wash system is largely suspended solids, with a small amount of residual dissolved solids. The hydrolysis of this glucan-rich material (i.e., the cellulose-rich solids stream) may be accomplished in an agitated, pH-controlled and temperature-controlled hydrolyzer, separate from the hydrolysis of the dissolved solids fraction of the wash. As noted above, the isolation and independent hydrolysis of the hemicellulosic oligomers allows for a better function of the glucanase applied to the cellulose-rich solids stream.

While glucan is the predominant sugar polymer in the fibrous cellulose-rich solids stream, xylan is still generally present. The resulting hydrolysate sugar stream is then a mixture of glucose, glucose oligomers, hemicellulose monomers, and hemicellulose oligomers. When the starting feedstock is hardwood or an agricultural residue such as sugarcane bagasse or straw, the hemicellulose is primarily xylose/xylan. When the starting feedstock is a softwood, the hemicellulose includes significant C₆ sugars, such as mannose/mannan.

In some embodiments, residual suspended solids are separated (such as with a horizontal decanter), re-suspended in process water, and then given an additional dose of enzymes. The purpose of this second hydrolysis is to complete the conversion of glucan by maintaining a glucose concentration low enough that glucose inhibition is not encountered (such as less than 55 g/L).

Step (g) may include fermentation of the glucose to any fermentation product. In some embodiments, step (g) comprises fermentation of the hemicellulosic monomers to any fermentation product which may be the same or different than the glucose-fermentation product. In some embodiments, step (g) comprises concentration of the glucose and/or the hemicellulosic monomers by evaporation, membrane filtration, or another suitable operation.

Optionally, the process further comprises removing at least a portion of the lignin, in dissolved form, from the extract liquor. Optionally, the process further comprises removing lignin that is present in suspended form from the extract liquor.

Other variations of the invention provide a process for producing fermentable sugars from cellulosic biomass, the process comprising:

(a) providing a feedstock comprising cellulosic biomass;

(b) optionally pre-steaming the feedstock;

(c) extracting the feedstock with liquid hot water under effective extraction conditions to produce cellulose-rich solids and an extract liquor containing dissolved solids, wherein the dissolved solids include hemicellulosic oligomers and lignin;

(d) washing the cellulose-rich solids to produce a washed cellulose-rich solids stream comprising glucan;

(e) hydrolyzing the hemicellulosic oligomers to hemicellulosic monomers by contacting the extract liquor with an acid catalyst or enzymes possessing hemicellulase activity;

(f) hydrolyzing the glucan to glucose by contacting the cellulose-rich solids stream with an acid catalyst or enzymes possessing glucanase activity, wherein step (f) includes removing the glucose in situ by microfiltration and/or ultrafiltration and/or other separating means; and

(g) recovering, fermenting, and/or further processing each of the hemicellulosic monomers and the glucose, separately or in combination.

The in situ removal of glucose during step (f), by microfiltration, ultrafiltration, or other means, allows for high solids loadings of glucan since product inhibition is avoided or reduced. In the absence of high concentrations of hemicellulose oligomers (due to the washing step), the glucanase remains active, producing glucose for a longer time. This allows for the possibility of running a fed batch with a much higher effective solids loading, when the glucose is removed from the hydrolyzer to prevent glucose inhibition, such as via in situ removal by microfiltration and/or ultrafiltration.

In some embodiments, effective extraction conditions include an extraction temperature selected from about 160° C.-220° C., about 160-200° C., or about 170-185° C. and an extraction time selected from about 3 minutes to about 4 hours, about 3-30 minutes, or about 10-20 minutes. An organic or inorganic acid catalyst may be utilized to aid extraction.

In some embodiments, washing in step (d) removes hydrolysis inhibitors from the cellulose-rich solids and generates a wash liquid that is combined with the extract liquor. The hydrolysis inhibitors may include one or more compounds selected from the group consisting of acetic acid, formic acid, lactic acid, furfural, hydroxymethylfurfural, hemicellulose oligomers, and combinations, derivatives, or degradation products thereof. Washing may be performed at a washing pH is selected from about 3 to about 7, or about 5 to about 6 in certain embodiments. Washing may be performed countercurrently.

Steps (e) and (f) may be performed separately or in a combined process step or unit. In some embodiments, the microfiltration and/or ultrafiltration generates a retentate that is returned to step (f) to recycle the acid catalyst or the enzymes possessing glucanase activity.

In some embodiments, step (g) comprises fermentation of the glucose and/or fermentation of the hemicellulosic monomers. Step (g) may include concentration of the glucose and/or the hemicellulosic monomers by evaporation, membrane filtration, and/or another unit operation.

The process may further include lignin removal and recovery. Preferably, the process includes generation of a cake of the residual lignin solids in the hydrolysate with a total solids content greater than about 25 wt %, 30 wt %, 35 wt %, 40 wt %, 45 wt %, 50 wt % or higher. High solids levels make it feasible for combustion in a biomass boiler with no further moisture removal.

Other variations of the invention provide a process for producing fermentable sugars from sugarcane bagasse, sugarcane straw, energy cane bagasse and/or energy cane straw, the process comprising:

(a) providing a feedstock comprising sugarcane bagasse, sugarcane straw, energy cane bagasse and/or energy cane straw;

(b) optionally cleaning and/or pre-steaming the feedstock;

(c) extracting the feedstock with liquid hot water under effective extraction conditions to produce cellulose-rich solids and an extract liquor containing dissolved solids, wherein the dissolved solids include hemicellulosic oligomers and lignin;

(d) washing the cellulose-rich solids to produce a washed cellulose-rich solids stream comprising glucan;

(e) hydrolyzing the hemicellulosic oligomers to hemicellulosic monomers by contacting the extract liquor with an acid catalyst or enzymes possessing hemicellulase activity;

(f) hydrolyzing the glucan to glucose by contacting the cellulose-rich solids stream with an acid catalyst or enzymes possessing glucanase activity, wherein step (f) includes removing the glucose in situ by microfiltration and/or ultrafiltration; and

(g) recovering, fermenting, and/or further processing each of the hemicellulosic monomers and the glucose, separately or in combination.

Other variations of the invention provide a process for producing fermentable sugars from sugarcane bagasse, sugarcane straw, energy cane bagasse and/or energy cane straw, the process comprising:

(a) cleaning a feedstock comprising sugarcane bagasse, sugarcane straw, energy cane bagasse and/or energy cane straw to remove sand, dirt, little rocks, and non-cellulosic debris;

(c) pre-steaming the feedstock;

(c) extracting the feedstock with liquid hot water under effective extraction conditions to produce cellulose-rich solids and an extract liquor containing dissolved solids, wherein the dissolved solids include hemicellulosic oligomers and lignin;

(d) washing the cellulose-rich solids to produce a washed cellulose-rich solids stream comprising glucan;

(e) hydrolyzing the hemicellulosic oligomers to hemicellulosic monomers by contacting the extract liquor with an acid catalyst or enzymes possessing hemicellulase activity;

(f) hydrolyzing the glucan to glucose by contacting the cellulose-rich solids stream with an acid catalyst or enzymes possessing glucanase activity, wherein step (f) includes removing the glucose in situ by microfiltration and/or ultrafiltration;

(g) fermenting the hemicellulosic monomers to ethanol or another fermentation product; and

(h) fermenting the glucose to ethanol or another fermentation product, which may be the same product or a different product than that generated in step (g).

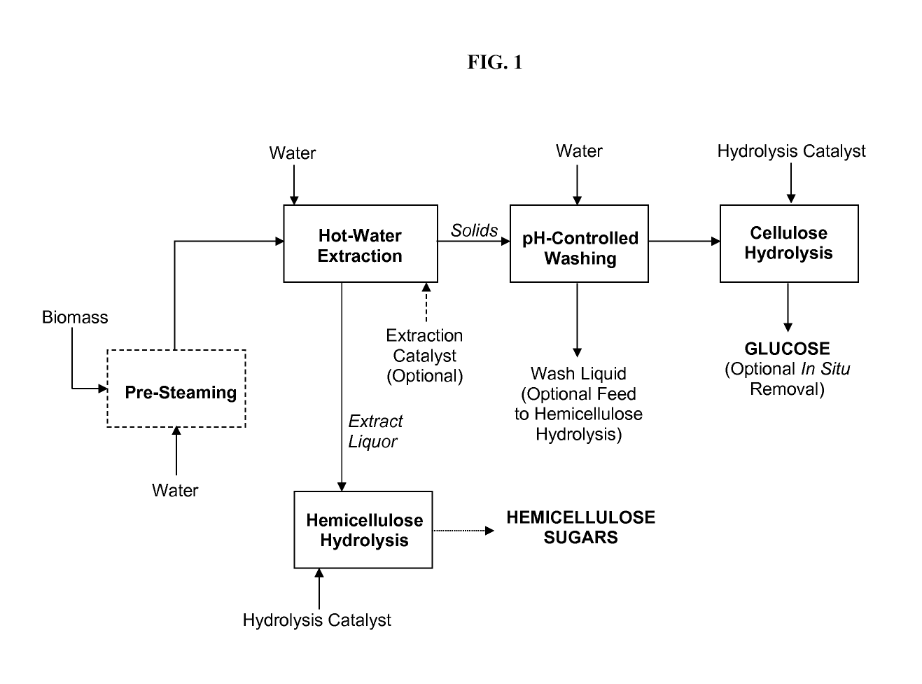

FIG. 1 is an exemplary block-flow diagram of the process and system of the invention, in some embodiments. In FIG. 1, cellulosic biomass is optionally pre-steamed before feeding to a hot-water extraction unit. This unit is configured for extracting the biomass (and/or pre-steamed biomass) with liquid hot water under effective extraction conditions to produce cellulose-rich solids and an extract liquor containing dissolved solids, with hemicellulosic oligomers and lignin. An extraction catalyst is optional. The solids are then sent to a pH-controlled washing unit configured to produce a washed cellulose-rich solids stream comprising glucan. The washing is carried out at a washing pH selected from about 3 to about 7, such as about 5 to about 6. The washing removes hydrolysis inhibitors from the cellulose-rich solids. Optionally, the wash liquid may be combined with another stream in the process, such as the feed to the hemicellulose hydrolysis reactor (e.g., when the wash liquid contains high concentrations of xylose oligomers). The washed glucan-rich solids are hydrolyzed with an acid or enzyme catalyst (or a base catalyst) to produce glucose in solution. The hemicellulosic oligomers are hydrolyzed to hemicellulosic monomers by contacting the extract liquor with enzymes possessing hemicellulase activity, or with an acid (or base) catalyst.

In some embodiments, the process starts as biomass is received or reduced to approximately ¼″ thickness. In a first step of the process, the biomass is fed to a pressurized extraction vessel operating continuously or in batch mode. The biomass may be pre-steamed or water-washed to remove dirt and entrained air. The biomass is immersed with aqueous liquor or saturated vapor and heated to a temperature between about 100° C. to about 250° C., for example 150° C., 160 C., 170° C., 180° C., 190° C., 200° C., or 210° C. Preferably, the biomass is heated to about 180° C. to 210° C. The pressure in the pressurized vessel may be adjusted to maintain the aqueous liquor as a liquid, a vapor, or a combination thereof. Exemplary pressures are about 1 atm to about 30 atm, such as about 3 atm, 5 atm, 10 atm, or 15 atm.

The aqueous liquor may contain acidifying compounds, such as (but not limited to) sulfuric acid, sulfurous acid, sulfur dioxide, acetic acid, formic acid, or oxalic acid, or combinations thereof. The dilute acid concentration can range from 0.01% to 10% as necessary to improve solubility of particular minerals, such as potassium, sodium, or silica. Preferably, the acid concentration is selected from about 0.01% to 4%, such as 0.1%, 0.5%, 1%, 1.5%, 2%, 2.5%, 3%, or 3.5%.

The acid for hemicellulose hydrolysis may be selected from sulfuric acid, sulfurous acid, or sulfur dioxide. Alternatively, or additionally, the acid may include formic acid, acetic acid, or oxalic acid from the cooking liquor or recycled from previous hydrolysis. Alternatively, hemicellulase enzymes may used for hemicellulose hydrolysis.

When enzymes are employed for the cellulose hydrolysis, the enzymes are preferably cellulase enzymes. Enzymes may be introduced along with the wash solution, e.g. water, recycled condensates, recycled permeate, or combinations thereof. Alternatively, or additionally, enzymatic hydrolysis may be carried out following washing and removal of hemicelluloses, minerals, and other soluble material.

When an acid is employed for the cellulose hydrolysis, the acid may be selected from sulfuric acid, sulfurous acid, sulfur dioxide, formic acid, acetic acid, oxalic acid, or combinations thereof. Dilute-acid hydrolysis is preferred, to avoid sugar degradation. Acids may be introduced to the extracted chips along with the wash solution, e.g. water, recycled condensates, recycled permeate, or combinations thereof. Alternatively, or additionally, acid hydrolysis may be carried out following washing and removal of hemicelluloses, minerals, and other soluble material.

A process step may include drying of the extracted material to a desired final moisture. The heat necessary for drying may be derived from combusting part of the starting biomass. Alternatively, or additionally, the heat for drying may be provided by other means, such as a natural gas boiler or other auxiliary fossil fuel, or from a waste heat source.

Another process step may include preparing the residual solids (high in lignin) for combustion. This step may include refining, milling, fluidizing, compacting, and/or pelletizing the dried material. The solids may be fed to a boiler in the form of fine powder, loose fiber, pellets, briquettes, extrudates, or any other suitable form.

Another process step may include evaporation of hydrolysate to remove some or most of the volatile acids. The evaporation step is preferably performed below the acetic acid dissociation pH of 4.8, and most preferably a pH selected from about 1 to about 2.5. In some embodiments, additional evaporation steps may be employed. These additional evaporation steps may be conducted at different conditions (e.g., temperature, pressure, and pH).

In certain embodiments, the process further comprises combining, at a pH of about 4.8 to 10 or higher, a portion of the vaporized acetic acid with an alkali oxide, alkali hydroxide, alkali carbonate, and/or alkali bicarbonate, wherein the alkali is selected from the group consisting of potassium, sodium, magnesium, calcium, and combinations thereof, to convert the portion of the vaporized acetic acid to an alkaline acetate. The alkaline acetate may be recovered. If desired, purified acetic acid may be generated from the alkaline acetate.

In some embodiments, some or all of the organic acids evaporated may be recycled, as vapor or condensate, to the first step (cooking step) and/or third step (washing step) to remove assist in the removal of minerals from the biomass. This recycle of organic acids, such as acetic acid, may be optimized along with process conditions that may vary depending on the amount recycled, to improve the cooking and/or washing effectiveness.

Some embodiments of the invention enable processing of “agricultural residues,” which for present purposes is meant to include lignocellulosic biomass associated with food crops, annual grasses, energy crops, or other annually renewable feedstocks. Exemplary agricultural residues include, but are not limited to, corn stover, corn fiber, wheat straw, sugarcane bagasse, rice straw, oat straw, barley straw, miscanthus, energy cane, or combinations thereof. In certain embodiments, the agricultural residue is sugarcane bagasse.

Optionally, some or all of the hemicellulosic sugars are combined with the glucose, to form a combined biomass-sugars stream. In some embodiments, the hemicellulosic sugars are separately recovered from the glucose. In some embodiments, fermentable hemicellulose sugars are recovered from solution, in purified form.

In some embodiments, glucose and/or fermentable hemicellulose sugars are fermented to produce of biochemicals or biofuels such as (but by no means limited to) ethanol, 1-butanol, isobutanol, acetic acid, lactic acid, or any other fermentation products. A purified fermentation product may be produced by distilling the fermentation product, which will also generate a distillation bottoms stream containing residual solids. A bottoms evaporation stage may be used, to produce residual solids.

Part or all of the residual solids may be co-combusted with biomass residual solids (e.g., lignin), if desired. Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

U.S. Patent App. Publication No. 2011/0081689 to Flanegan et al., published Apr. 7, 2011, is incorporated by reference herein. Some embodiments of the present invention may optionally incorporate additional steps or conditions (such as, but not limited to, mechanical treatments) that are disclosed in U.S. Patent App. Publication No. 2011/0081689.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing fermentable sugars from cellulosic biomass, said process comprising:
(a) providing a feedstock comprising cellulosic biomass;
(b) optionally pre-steaming said feedstock;
(c) extracting said feedstock with liquid hot water under effective extraction conditions to produce cellulose-rich solids and an extract liquor containing dissolved solids, wherein said dissolved solids include hemicellulosic oligomers and lignin;
(d) washing said cellulose-rich solids to produce a washed cellulose-rich solids stream comprising glucan;
(e) hydrolyzing said hemicellulosic oligomers to hemicellulosic monomers by contacting said extract liquor with an acid catalyst or enzymes possessing hemicellulase activity;
(f) hydrolyzing said glucan to glucose by contacting said cellulose-rich solids stream with an acid catalyst or enzymes possessing glucanase activity, wherein step (f) includes removing said glucose in situ by microfiltration and/or ultrafiltration; and
(g) recovering, fermenting, and/or further processing each of said hemicellulosic monomers and said glucose, separately or in combination.

2. The process of claim 1, wherein said effective extraction conditions include an extraction temperature selected from about 160° C. to about 220° C., and an extraction time selected from about 3 minutes to about 4 hours.

3. The process of claim 2, wherein said effective extraction conditions include an extraction temperature selected from about 160° C. to about 200° C., and an extraction time selected from about 3 minutes to about 30 minutes.

4. The process of claim 3, wherein said effective extraction conditions include an extraction temperature selected from about 170° C. to about 185° C., and an extraction time selected from about 10 minutes to about 20 minutes.

5. The process of claim 1, wherein said extracting in step (c) employs an acid catalyst.

6. The process of claim 5, wherein said acid catalyst is an organic acid.

7. The process of claim 6, wherein said organic acid is acetic acid.

8. The process of claim 5, wherein said acid catalyst is an inorganic acid.

9. The process of claim 8, wherein said inorganic acid is sulfuric acid, sulfurous acid, sulfur dioxide, or a combination thereof.

10. The process of claim 1, wherein said washing in step (d) removes hydrolysis inhibitors from said cellulose-rich solids and generates a wash liquid that is combined with said extract liquor.

11. The process of claim 10, wherein said hydrolysis inhibitors include one or more compounds selected from the group consisting of acetic acid, formic acid, lactic acid, furfural, hydroxymethylfurfural, hemicellulose oligomers, and combinations, derivatives, or degradation products thereof.

12. The process of claim 1, wherein said washing pH is selected from about 3 to about 7.

13. The process of claim 12, wherein said washing pH is selected from about 5 to about 6.

14. The process of claim 1, wherein step (d) employs countercurrent washing.

15. The process of claim 1, wherein steps (e) and (f) are conducted separately.

16. The process of claim 1, wherein said microfiltration and/or ultrafiltration generates a retentate that is returned to step (f) to recycle said acid catalyst or said enzymes possessing glucanase activity.

17. The process of claim 1, wherein step (g) comprises fermentation of said glucose and/or fermentation of said hemicellulosic monomers.

18. The process of claim 1, wherein step (g) comprises concentration of said glucose and/or said hemicellulosic monomers by evaporation.

19. The process of claim 1, wherein step (g) comprises concentration of said glucose and/or said hemicellulosic monomers by membrane filtration.

20. The process of claim 1, said process further comprising removing at least a portion of said lignin from said extract liquor.
